(19) United States
(12) Patent Application Publication
(10) Pub. No.: US 2015/0203805 A1
(43) Pub. Date: Jul. 23, 2015

(54) EFFLUENT MONITORING SYSTEMS

(71) Applicant: Bactest Limited, Cambridge (GB)

(72) Inventor: Derek Price, Cambridge (GB)

(21) Appl. No.: 14/423,168

(22) PCT Filed: Aug. 16, 2013
(86) PCT No.: PCT/GB2013/052178

§ 371 Date: Feb. 23, 2015

(30) Foreign Application Priority Data
Aug. 22, 2012 (GB) 1214966.2

(51) Int. Cl. C12M 1/34; B01L 1/00; C12M 1/00; G01N 33/18

(52) U.S. Cl. CPC C12M 41/40; G01N 33/1806; B01L 1/00; C12M 23/28; C12M 23/38

(57) ABSTRACT

We describe a system for monitoring effluent discharge to determine one or both of a biochemical oxygen demand (BOD) and a toxicity of the discharge. The system comprises: a culture vessel comprising a sealable chamber for culturing a fluid sample and a pressure measurement transducer for measuring a pressure in a headspace of the chamber; and a data processing system to: input pressure data from the pressure measurement transducer; and determine a value for one or both of BOD and toxicity from the change in pressure measured by the pressure transducer.

### FIELD OF THE INVENTION

This invention relates to methods and systems for monitoring effluent discharge, in particular for biochemical oxygen demand (BOD) and/or toxicity.

### BACKGROUND TO THE INVENTION

Food and other manufacturing plants, abattoirs and the like may discharge into water courses. It is important to ensure that this discharge is within acceptable/legal limits, both to protect the environment and to comply with legislation. If it is found that effluent scheduled for discharge is outside tolerance limits then the effluent can be treated; suitable chemicals are available for purchase.

In the UK there is a standard test known as the BOD5 (biological oxygen demand 5 day test) which can be employed to test a water sample, in particular to determine the biodegradable pollution load. However, as the name implies, this involves incubating a sample over 5 days to characterise the sample by its oxygen use, which is obviously inconvenient in practice. The output data from a BOD5 test may be, for example, expressed as mg/L (milligrams per litre) of BOD (biodegradable) material. Tests are available to determine COD (chemical oxygen demand) which is representative of the total organic pollution load, and a COD test may be faster. However the industry standard is the BOD5 test and when monitoring effluent discharge it is the biological oxygen demand on which emphasis is placed.

A further problem with monitoring effluent discharge can arise when, for example, the discharge is toxic to the activated sludge in sewage plants: managing a waste water treatment plant can be difficult and a related problem exists in identifying effluents which could adversely affect the operation of such plants.

### SUMMARY OF THE INVENTION

According to the present invention there is therefore provided a system for monitoring effluent discharge to determine one or both of a biochemical oxygen demand (BOD) and a toxicity of said discharge, the system comprising: a culture vessel comprising a sealable chamber for culturing a fluid sample and a pressure measurement transducer for measuring a pressure in a headspace of said sealable chamber; and a data processing system to: input pressure data from said pressure measurement transducer; and determine a value for one or both of said BOD and said toxicity from said change in pressure measured by said pressure transducer.

Embodiments of the above described system can provide a value which maps or is calibrated to the value which would be obtained from a standard BOD5 test, which is surprising as the growth/metabolism of bacteria, protozoa and the like both uses gas (oxygen) and produces gas (CO₂) within the sealed chamber. In embodiments of the system a pressure drop is measured and, without wishing to be bound by theory, the overall drop in pressure is believed to relate to the overall production of bacteria, in part from the gas in the headspace (without this one might expect that the gas use and gas production would approximately balance).

In some preferred implementations of the system the culture vessel includes a magnetically driven paddle blade or propeller to promote gas exchange between the liquid phase and headspace (the magnetic drive conveniently facilitating agitation round the chamber is sealed). In embodiments of the system this or another means may be provided to aerate the fluid sample prior to sealing the chamber to provide a common base line gas level when starting the procedure (to avoid effects which can otherwise be seen due to restriction in bacterial growth due to gas depletion). Such a process may be part of a control protocol implemented by software running on the device. Similarly device software may also disregard an initial phase of pressure drop, for example over an initial period of less than 30 minutes, during which unreliable readings can often be obtained.

Preferred implementations of the system also incorporate temperature control again, for example, implemented by the system control software of the device. The temperature may be controlled to a defined, calibration value or, for example, to the temperature of the tank from which the effluent is being discharged, or to some other value, for example the temperature of the water course into which the discharge is made or the temperature of a water treatment plant for the effluent.

Where the system is being used to monitor effluent for toxicity, in embodiments the discharge fluid sample is diluted (with water) prior to making a measurement. This is because toxic materials in the fluid sample can otherwise effect the bacterial growth, which can lead to unreliable results. Dilution reduces the effective toxicity; the dilution prior to incubation may be to a high degree, for example at least 90%, 95%, 98% or 99% more dilution (that is, leaving 10% or less of the original sample). In embodiments this dilution may be performed automatically by the system or it may be part of an operation or protocol through which a user is taken, for example by displaying or otherwise presenting instructions to a user defining steps in operation of the device.

In embodiments the biochemical oxygen demand and/or toxicity may be determined either by an absolute pressure drop or, more preferably, by a rate of pressure drop (the BOD/toxicity value may correspond to or be dependent upon this rate). For example the pressure drop per 10, 20, 30, 40, 50 or 60 minutes may be determined. In embodiments the system may be calibrated by matching the rate of pressure drop to a BOD and/or toxicity level, for example determined based upon a determined straight light (linear) relationship and/or a calibration curve. Additionally or alternatively, however, the system may determine an absolute pressure drop and/or an integrated pressure drop (the area under a pressure-time curve). In some embodiments the overall measurement may be relatively quick, for example less than 6, 4, 3, 2 or 1 hour.

Preferably the or each culture vessel is removable for disposal. Further, a culture vessel may be provided as a separate item of commerce, in particular including one or more types of bacteria/protozoa for culture. In such a case preferably the bacteria are provided within the culture vessel, and preferably the vessel and bacteria combination is sealed, for example in a feral envelope. The bacteria may be freeze dried.

In preferred embodiments a pair of culture vessels is provided, one to act as a control. With this arrangement, preferably bacteria for the culture test and control vessels are provided together, for example in a pair of joined packets. Where the bacteria are provided within a culture vessel, a pair of single culture vessels may be provided, preferably each within a respective sealed enclosure, in a single package or packet. In this way the bacteria are stored together so that they should age at similar rates. Preferably the bacteria are also derived from the same original batch.

In embodiments, in particular for toxicity testing, the bacteria may be derived from returned activated sludge from a waste water treatment plant. Optionally the bacteria may be derived from the particular waste water treatment plant with which the effluent will eventually be treated, since the populations of bacteria can vary from one plant to another. For example bacteria may be obtained from a plant, carefully dried using a microwave oven, and then a defined weight of bacteria added to a culture and/or control vessel.

The bacteria in different parts of a waste water treatment plant can be of different types of species. Thus at the front end of a plant. The bacteria may tend to be carbonaceous, whereas at the far end of a plant (typically the end from which RAS may be derived) the bacteria are more likely to be nitrogenous. Thus embodiments of the system may employ one or other or both of these types of bacteria either together or separately. For example separate tests for either BOD or toxicity may be applied using bacteria predominantly of one or more different types such as carbonaceous, nitrogenous and the like. Optionally an operational protocol may involve taking bacteria from RAS from a waste water treatment plant to be used to treat the effluent at intervals over a time period since the constitutional population of bacteria in a plant can also vary over time. Depending upon the test applied, in some embodiments the quantity of bacteria employed may be such that an excess of bacteria is present, that is substantially more bacteria than would in principle be needed to metabolise the ‘food’ in the liquid sample. This can also be advantageous since, even with a relatively low level of food (dilute effluent) a relatively significant quantity of gas may be consumed, thus facilitating measurement.

The data processing system may be implanted in hardware, in software or using a combination of the two. Thus, for example, the data processing system may comprise a micro processor coupled to working memory and to program memory storing processor control code for a procedure to implement the described systems/methods. In embodiments a removable, non-volatile memory card is provided for the system to facilitate extraction of the data. preferably a system also includes a communications interface for uploading either the raw pressure data, or data derived from this, or both, to a remote data management computer system. In embodiments this may be implemented by a computer network connection (wired and/or wireless). The remote data management computer system may comprise, for example, a laptop computer. In this way data from one or more of the above described systems/devices can be uploaded and monitored to provide a management tool this may be employed, for example, to compare results between two or more systems, to track trends over time, for remote monitoring of multiple effluent discharges (especially in a large manufacturing plant), to provide all its data for compliance purposes, and the like.

In a related aspect there is provided a pair of sets of bacteria, each in a respective sealed enclosure, wherein said sealed enclosures are physically linked or packaged together.

In a further related aspect there is provided a method of evaluating effluent to determine one or both of a biochemical oxygen demand (BOD) and a toxicity of said effluent, the method comprising: obtaining a fluid sample from a fluid of said effluent; providing said fluid sample to a sealed test chamber such that said fluid sample incompletely fills said sealed test chamber leaving a headspace; incubating said fluid sample with test bacteria in said sealed test chamber; determining a change in pressure in said headspace during said incubating: and determining one or both of said BOD and said toxicity from said change in pressure.

### BRIEF DESCRIPTION OF THE DRAWINGS

These and other aspects of the invention will now be further described, by way of example only, with reference to the accompanying figures in which:

FIG. 1 shows a high level schematic diagram of a waste water treatment plant;

FIGS. 2a and 2b show a culture vessel for use in embodiments of the invention under, respectively normal atmospheric pressure and reduced pressure;

FIG. 3 shows the variation of pressure with time when incubating influent over a period of hours;

FIG. 4 shows a variation of pressure with time for different ratios of sample to headspace volume;

FIGS. 5a and 5b show, respectively, variation of pressure with time for a toxic fluid and a control, and for different toxicity levels; and

FIG. 6 shows a portable trade effluent monitoring system according to an embodiment of the invention.

### DETAILED DESCRIPTION OF PREFERRED EMBODIMENTS

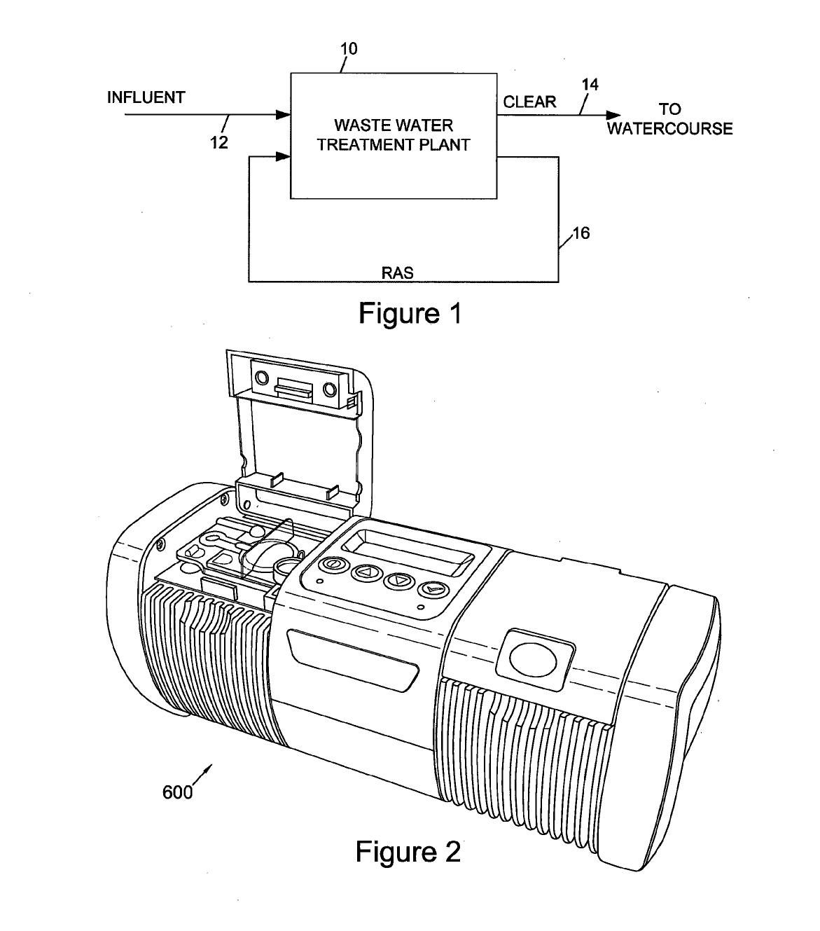

FIG. 1 shows, at a high level, a schematic diagram of the operation of a waste water treatment plant 10. Thus the plant accepts influent 12, fluid from which the solids have been substantially removed, containing a high level of ‘food’ for bacteria, protozoans and the like (biomass') and having a high biochemical oxygen demand (BOD). The output from the plant has two components, a clear component 14 which may be provided to a water course and a biological component 16 comprising living biological material referred to as returned activated sludge (RAS), typically at around 60% concentration. The RAS is provided back to the input side of the plant to help maintain the eco system.

We have previously described a system for monitoring the metabolism/growth of microorganisms, the system comprising a sealed chamber with a flexible diaphragm to provide sensitive pressure measurements of gas pressure in the headspace above a culture liquid. For details reference may be made, for example, to US2005/0170497 (incorporated by reference).

The inventors have carried out experimental work on the suitability of such a system for application to various effluent-related fluids.

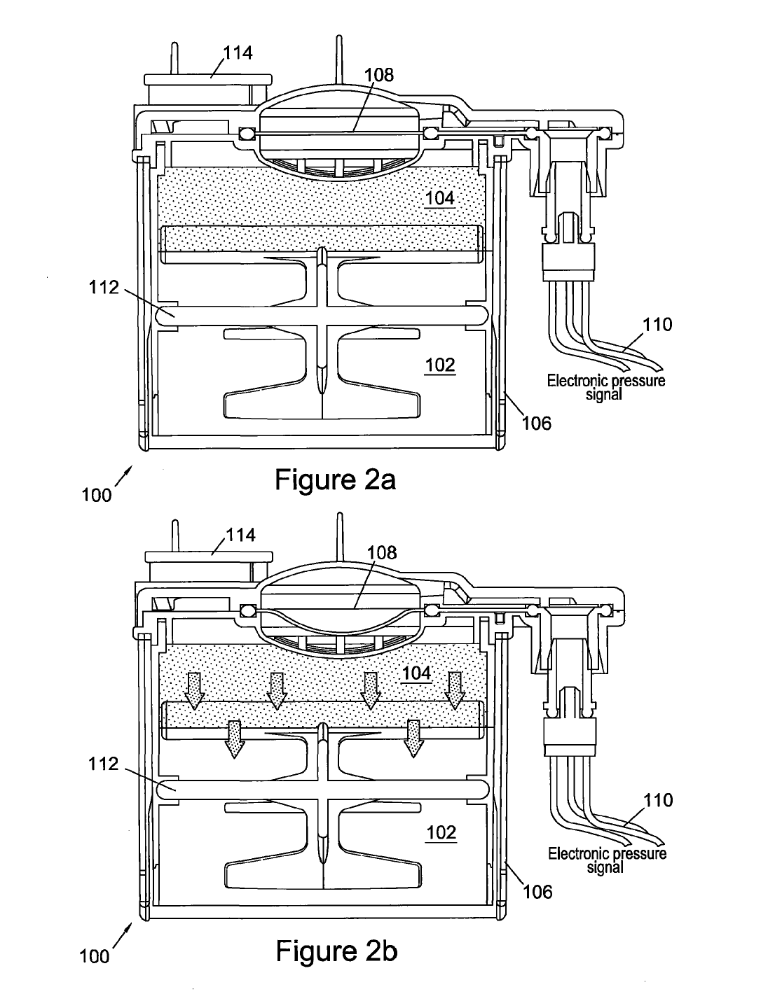

FIGS. 2a and 2b show, schematically, an embodiment of a similar device 100 under, respectively, normal atmospheric pressure and negative pressure (in operation either negative pressure or positive pressure may be produced). Thus a culture 102 of biological material undergoes metabolism and growth during which it exchanges gases with the aqueous liquid (water) carrying cells depending upon various factors gas may be used and/or produced, for example the cells may produce carbon dioxide during respiration. A gaseous headspace 104 of the sealed culture chamber 106 thus experiences changes in pressure due to exchange of gas with the culture medium, and these are monitored by a diaphragm 108 and converted to an electronic pressure signal 110 which may, for example, be digitised and processed electronically by hardware, software or a combination of the two. Preferably the system also includes an agitator 112 and temperature control (not shown), as well as a sealable inlet/outlet port 114.

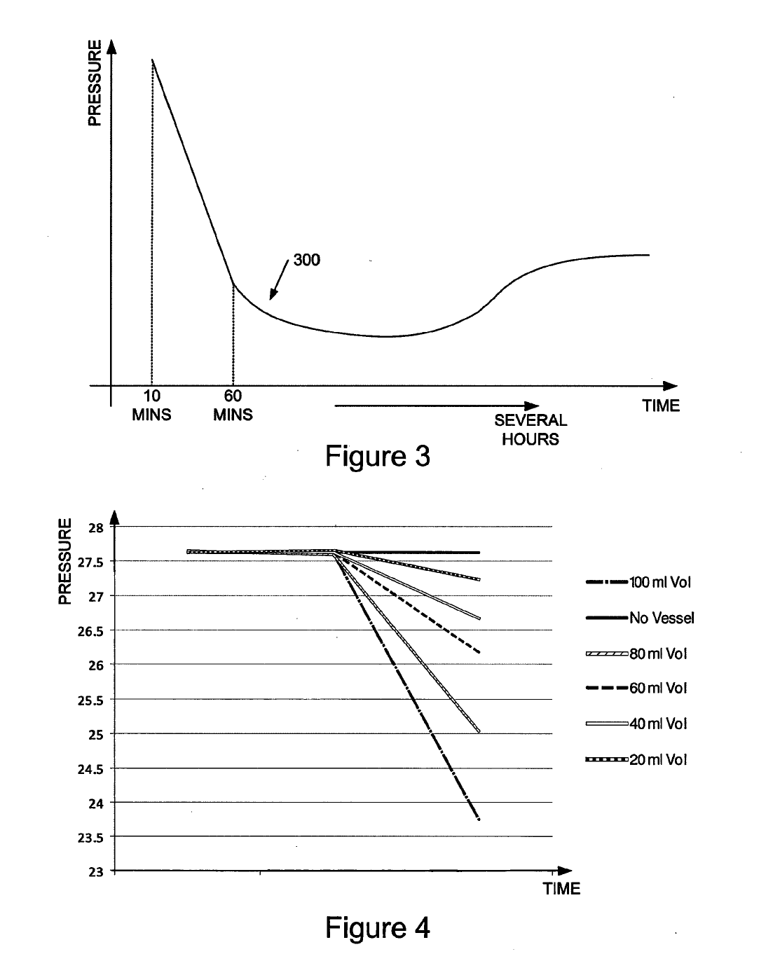

Experiments were performed to determine what parameters can be measured by apparatus of the type illustrated in FIG. 2. Initial experiments determined that the general shape of a pressure-time curve for incubated fluid is as illustrated in FIG. 3. Thus there is an initial period during which the pressure can vary and results appear unreliable. This typically lasts up to around 10 minutes. The pressure then begins to fall, flattening out in a trough region 300 after around one to a few hours. Over a further period of several hours the pressure then gradually starts to rise once more (the graph of FIG. 3 is not to scale). The initial rate of pressure drop appears to be related to the concentration of food in the influent, a faster drop being observed with more food present. Broadly, the pressure drop per hour correlates with the amount of available food. Here ‘food’ is used to describe material in all forms which facilitate the growth of bacteria (including, for example, more or less complex carbon sources, sources of oxygen, nitrogen, phosphorous, and ammonia, and also including, potentially, other bacteria). It is surmised that the pressure drop relates to the conversion of gas into living biomass since although oxygen is used during bacterial growth, carbon dioxide is produced. It is further surmised that the trough region occurs when the oxygen has been depleted, the subsequent smaller pressure rise relating to anaerobic respiration producing carbon dioxide. However the inventor does not wish to be bound by theory.

### Sample to Headspace Ratio

An experiment was performed to investigate the effect of the sample to headspace ratio in the sealed culture vessel. This showed that the liquid phase (sample) to gaseous phase (measured head space) volume ratio can be used to adjust the sensitivity of the test system.

One experimental protocol was as follows:
1. Fresh, settled (solids removed) influent was stored overnight at 4-8 Deg C. without aeration. A (normal) small amount of floating solids remained but very minor.
2. Fresh RAS (return activated sludge) was stored overnight at 4-8 Deg C. with aeration.
3. Influent was equilibrated to 20 deg C.
4. RAS was equilibrated to 20 deg C., washed 3 times in clean water and mixed 1:1 with Influent.
5. RAS/Influent mixture was added to culture vessels at varying volumes and mixed for 5 minutes open to the air.
6. Vessel sealed and logging started in bench rig.

FIG. 4 shows the variation of pressure with time with different sample volumes:

Varying sample volume to headspace ratio gave significantly different pressure drop results, and the variation was reasonably consistent. A ratio of ˜1:1 was found to be useful for the particular development rig employed, with a working volume of ˜100 ml—but the skilled person will appreciate that this is particular to the rig employed. More importantly the experiments showed that the liquid phase to gaseous phase volume ratio is one easily modified parameter that can be adjusted to affect the rate of pressure change. This shows that test protocol may be modified to account for different test conditions and sensitivity requirements (within limits) if desired.

### Measuring the Effects of Toxic Waste Materials

These experiments showed that the metabolic activity of microbes in the activated sludge is adversely affected by toxic materials entering the process in the effluent. This in turn can be measured as a function of differences in pressure drop due to metabolic activity, providing a method of monitoring toxic events that might be highly detrimental to the process.

One experimental protocol to investigate toxicity was as follows:
1. Fresh, settled (solids removed) influent was stored overnight at 4-8 Deg C. without aeration. Note: a (normal) small amount of floating solids remained but very minor.
2. Fresh RAS (return activated sludge) was stored overnight at 4-8 Deg C. with aeration.
3. Influent was equilibrated to 20 deg C.
4. RAS was equilibrated to 20 deg C., unmixed but in large surface area vessel, shaken every 15 minutes.
5. 30 ml RAS added to culture vessels and mixed for 15 minutes open to the air.
6. 30 ml Diluted Influent sample added to culture vessels
7. A 1 ml volume of diluted hypochlorite was added to one vessel, the other acted as a control
8. The two chambers were mixed for 3 minutes open to the air.
9. Vessels were sealed and logging started

In alternative protocols sodium azide, a metabolic inhibitor, may be employed rather than hypochlorite (as sodium azide is more chemically stable).

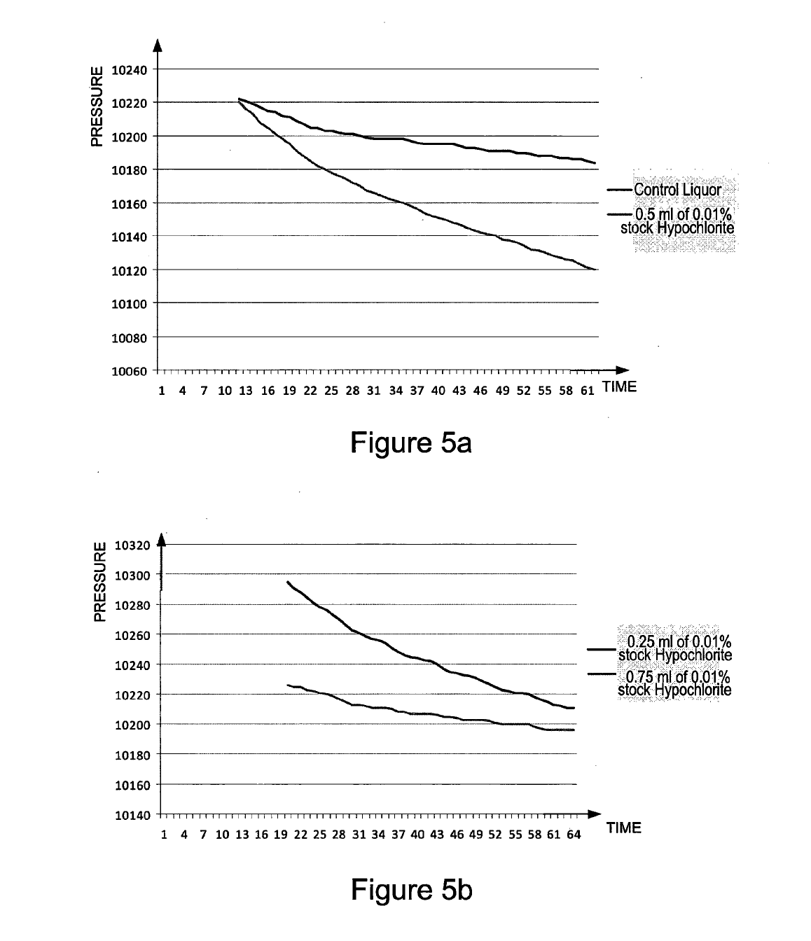

FIG. 5 shows graphs of the variation of pressure with time with a control, and with varying degrees of toxicity (using hypochlorite at 0.25 and 0.75 ml of 0.01% stock solution). It can be seen that there is a significantly different pressure drop in the test vessel when compared to the control, and that increasing concentration of toxic substance adversely affects metabolism and pressure drop. The level of toxic material can be measured by pressure change in systems of the type we describe.

Further experiments showed that apparatus of the type illustrated in FIG. 2 can be used as a proxy for a BOD5 measurement, determining the overall gas input/output balance for a given sample of fluid containing biomass over time and consistent with the food availability, but in around an hour as compared with 5 days.

In both BOD and toxicity tests dilution of samples can be useful to suppress bacterial growth limitations from toxic or inhibitory components/effects which can otherwise interfere with obtaining accurate results. Choosing a suitable level of dilution was found to be important in practice and thus an initial step of characterising the effluent to be monitored can be useful. In general the optimum degree of dilution may vary from discharge to discharge. In some preferred implementations the incubated effluent sample is diluted by at least 100:1, in embodiments by 200:1, 300:1, 400:1 or 500:1

Similarly a pre-oxygenation step is also helpful to reduce the risk of a test being unduly influenced by an inherent oxygen level in a sample. More generally, a step to equilibrate gaseous composition of biologically active samples or to control the level of gas, in particular oxygen, in a sample is helpful. Temperature control is also useful, in part because of the varying gas-dissolving ability of water at different temperatures.

### EXAMPLE EMBODIMENT

FIG. 6 shows an example of a portable trade effluent monitoring system 600 according to an embodiment of the invention.

The system is designed for rapid detection of variance from compliancy with trade effluent discharge consents. In embodiments it provides an easy to use compact portable contamination test that is used to monitor food waste and the strength of effluent by delivering a BOD5 Proxy in less than 1 hour. Samples are taken on site and the test begins in-situ immediately. The system thus removes opportunities for sample degradation caused by delay in transportation to laboratories and erroneous results caused by loss of sample identity.

Should a parameter for BOD be breached the system provides a warning and/or control output, in particular an alarm, so that effluent can be sidelined and flagged for further analysis and treatment. The alarm can indicate contamination to a pre-determined protocol, which may be user-defined.

The system is a quantitative test that detects variances from pre-set acceptable effluent parameters. In more detail, in embodiments the system delivers a BOD5 Proxy in liquids and macerated solids by incubating effluent samples. Two samples are introduced into the two culture vessels and the operator presses the start button and no further operator input is then required. The control software operates the test and the system does not require trained a operator.

In embodiments the system is a single (test versus control), two chamber instrument. The system maintains culture conditions within purpose designed, disposable culture vessels. In embodiments each chamber is temperature controlled (in embodiments in the range 14 to 44 degrees) and includes magnetically-drive, paddle-based mixing for optimised growth and detection of microbial activity.

Samples are inoculated into the culture vessel and the system closely controls growth conditions to a pre-determined protocol. A robust, disposable 50 ml culture vessel eliminates the need for cleaning of potentially hazardous bottles. The system also employs pressure sensing using a non-invasive process, which isolates the sensors, but more importantly forms a barrier to protect both the biological culture and the operator.

In embodiments the system is a sensitive, precision microbial respirometer and detector of microbial activity. Detection of metabolic activity is determined by pressure transients relating to gaseous exchanges within a 50 ml closed culture vessel as a result of microbial respiration. The system has pressure sensing and mixing technology that efficiently homogenises culture conditions and rapidly converts gaseous exchange due to metabolic processes into detectable pressure transients. In embodiments the system measures both positive and negative pressure—which has the advantage that monitoring can be performed on a range of microbial processes reacting to differing conditions within the culture chamber.

In embodiments the system can be used independently or connected to a PC, in embodiments via a USB connection. Using the PC a quality assurance manager can design and download protocols to the system and upload test results for analysis and/or audit. The system also has a removable SD card for experimental results storage, so that a unit can be used on or close to the production floor. Connecting the system to a PC, for example a Windows (RTM) compatible laptop, enables experiment visualization in near real time. The remote PC also enables a customised test protocol to be designed and downloaded to the system. For example the system may be configured to implement a test for variance from compliancy with trade effluent discharge consents.

Embodiments of the system implement self monitoring and calibration. In embodiments the system is compact, weighing under 3 kilos, is portable and operates on 12 dc and a mains adapter.

Preferably the culture vessels are supplied sterile (gamma irradiated) in protective packaging. Embodiments of the two testing chambers and sealed, easy to populate, and suitable for use with a wide range of sample types.

Embodiments of the system may be employed for, inter alia: monitoring trade effluent; in the manufacture food, beverage, home, beauty, and/or pharmaceutical products; to monitor the manufacture of products where process discharge includes one or more of fats, oils, heavy metals, chemicals, and detergents; and to demonstrate compliance with discharge consents.

No doubt many other effective alternatives will occur to the skilled person. It will be understood that the invention is not limited to the described embodiments and encompasses modifications apparent to those skilled in the art lying within the spirit and scope of the claims appended hereto.

1. A system for monitoring effluent discharge to determine one or both of a biochemical oxygen demand (BOD) and a toxicity of said discharge, the system comprising:
a culture vessel comprising a sealable chamber for culturing a fluid sample and a pressure measurement transducer for measuring a pressure in a headspace of said sealable chamber; and
a data processing system to:
input pressure data from said pressure measurement transducer; and
determine a value for one or both of said BOD and said toxicity from said change in pressure measured by said pressure transducer.

2. A system as claimed in claim 1 comprising a pair of said culture vessels, wherein one said culture vessel is a control.

3. A system as claimed in claim 1 wherein said culture vessel is removable for disposal.

4. A system as claimed in claim 1 in combination with bacteria for said culture vessel, wherein one or both of said culture vessel and said bacteria are provided in a sealed enclosure.

5. A system as claimed in claim 4 in combination with bacteria for said culture vessel, wherein two sets of said bacteria are provided in respective sealed enclosures, one for a control culture, and wherein said sealed enclosures are physically linked together.

6. A system as claimed in claim 1 in combination with bacteria for said culture vessel, wherein said bacteria comprise bacteria of at least two different types, characteristic of different types of bacteria in returned activated sludge of a waste water treatment plant.

7. A system as claimed in claim 1 wherein said data processor configured to determine said value from one or both of a pressure drop and a rate of pressure drop in said headspace of said culture vessel.

8. A system as claimed in claim 7 further comprising a communications interface for uploading one or both of said pressure data and said value to a remote data management computer system.

9. A system as claimed in claim 1 wherein said value is calibrated to represent a value from a standard BOD5 test.

10. A pair of sets of bacteria for a system as recited in claim 1, said set of bacteria in a respective sealed enclosure, wherein said sealed enclosures are physically linked or packaged together.

11. A method of evaluating effluent to determine one or both of a biochemical oxygen demand (BOD) and a toxicity of said effluent, the method comprising:
obtaining a fluid sample from a fluid of said effluent;
providing said fluid sample to a sealed test chamber such that said fluid sample incompletely fills said sealed test chamber leaving a headspace;
incubating said fluid sample with test bacteria in said sealed test chamber;
determining a change in pressure in said headspace during said incubating: and
determining one or both of said BOD and said toxicity from said change in pressure.

12. A method as claimed in claim 11 further comprising evaluating a control fluid sample with control bacteria in a second, sealed control chamber, and determining said BOD and/or toxicity from a difference between a response of said test and control chambers, the method further comprising providing said test and control bacteria to said test and control chambers from a common source, after storing under common storage conditions.

13. A method as claimed in claim 11 wherein said test bacteria comprises RAS (returned activated sludge) bacteria.

14. A method as claimed in claim 11, wherein said test bacteria comprise one or both of nitrogenous bacteria and carbonaceous bacteria.

15. A method as claimed in claim 11, further comprising diluting said fluid sample prior to said incubating.

16. A method as claimed in claim 15 wherein said diluting comprises diluting by at least 90%, 95%, 98% or 99%.

17. A method as claimed in claim 11 further comprising aerating said fluid sample prior to said incubating.

18. A method as claimed in claim 11 wherein said change in pressure comprises a fall in pressure or a rate of drop in said pressure.

19. (canceled)
